(19) United States
(12) Patent Application Publication
(10) Pub. No.: US 2024/0188562 A1
(43) Pub. Date: Jun. 13, 2024

(54) DISINFECTANT COMPOSITION FOR AUTOMOTIVE CARE AND METHODS OF USE

(71) Applicant: 3M INNOVATIVE PROPERTIES COMPANY, St. Paul, MN (US)

(72) Inventors: DanLi WANG, St. Paul, MN (US); Brian P. DUTKIEWICZ, Irvine, CA (US); Narina Y. STEPANOVA, St. Paul, MN (US); Briklyn M. WUICH, St. Paul, MN (US); Bathsheba E.F. Chong Conklin, St. Paul, MN (US); Linda J. Murray, Woodbury, MN (US); Judd D. Olson, Deephaven, MN (US)

(21) Appl. No.: 18/283,396

(22) PCT Filed: Mar. 24, 2022
(86) PCT No.: PCT/IB2022/052726

§ 371 Date: Sep. 21, 2023

Related U.S. Application Data
(60) Provisional application No. 63/169,087, filed on Mar. 31, 2021.

(51) Int. Cl. A01N 33/12; A01N 25/04; A01P 1/00

(52) U.S. Cl. CPC A01N 33/12; A01N 25/04; A01P 1/00

(57) ABSTRACT

A disinfecting composition useful for care and protection of interior automotive surfaces. The composition includes a silicone package, typically including a silicone fluid, and a disinfectant, typically a quaternary ammonium compound.

### SUMMARY

In the wake of COVID-19 pandemic and heightened awareness of surface contamination, consumers are paying more attention to getting the inside of their automobiles both clean and sanitized/disinfected. Most importance is generally placed on disinfecting high touch surfaces, including the steering wheel, door handles, shift lever, any buttons or touch screens, wiper and turn signal stalks, passenger and driver door armrests, grab handles, seats, and seat adjusters. However, vehicle surfaces are not made to withstand a constant onslaught of harsh cleaning/disinfect products and can wear down if cleaned too often, especially leather seats.

Silicone-based automotive detailing and protectants commonly available in the marketplace serve to clean and protect automotive surfaces. The term “automotive protectants” is defined herein as products which are applied to vinyl, leather, plastics, rubber, and other interior surfaces of cars, trucks, boats, and other vehicles to improve the gloss of such surfaces. The silicone-based (e.g., organopolysiloxane, silicone emulsion, silicone oil) coating on the substrate can also protect the substrate surfaces from heat damage and/or fading to sun exposure. The protectants may include UV protectors and freshener.

To date, disinfecting an automobile interior requires a two-step process, with the interior surface first cleaned with a disinfectant product and subsequently detailed and/or protected or first detailed and subsequently disinfected. This typically results in either an inadequate detailing due to residue left by the disinfectant or inadequate disinfection based on inference from the detailing/protecting wipe down. The consumer would prefer to have disinfectant product that can also restore color/shine/protect and/or deodorize, leaving a fresh scent, all in one step. It is believed that, to date, this type of product is not available in the marketplace.

The present disclosure generally describes silicone emulsion-based disinfect solutions that comprise one or more disinfectants, typically quaternary ammonium compounds. The compositions disclosed herein are designed to disinfect automobile interior surfaces and simultaneously clean, shine and protect the surface, all in one step.

As used herein, the term “substantial” or “substantially” means with relatively minor fluctuations or aberrations from the stated property, value, range of values, content, formula, and the like, and does not exclude the presence of additional materials, broader range values, and the like which do not materially affect the desired characteristics of a given composition, article, product, or method.

Herein, the terms “comprises” and “includes” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to claims of the disclosure that may afford certain benefits, under certain circumstances. However, other claims may also be preferred, under the same or other circumstances. Furthermore, the recitation of one or more preferred claims does not imply that other claims are not useful and is not intended to exclude other claims from the scope of the disclosure.

In this application, terms such as “a”, “an”, and “the” are not intended to refer to only a singular entity but include the general class of which a specific example may be used for illustration. The terms “a”, “an”, and “the” are used interchangeably with the term “at least one”. The phrases “at least one of” and “includes at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5).

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

Features and advantages of the present disclosure will be further understood upon consideration of the detailed description as well as the appended claims.

### DETAILED DESCRIPTION

Various embodiments and implementations will be described in detail. These embodiments should not be construed as limiting the scope of the present application in any manner, and changes and modifications may be made without departing from the spirit and scope of the inventions. Further, only some end uses have been discussed herein, but end uses not specifically described herein are included within the scope of the present application. As such, the scope of the present application should be determined by the claims.

Disinfecting car care compositions of the present disclosure include a silicone package, water, and a disinfectant package. The silicone package may include one or more silicone emulsions. Suitable silicone emulsions are preferably (at present) non-ionic and typically include 50-90% silicone fluid in water and are made from silicone fluids having a viscosity of 350 cSt to 1000 cSt. Particularly suitable commercially available silicone emulsions include XIAMETER 0349 available from Dow, Midland, MI and SM 2163 available from Momentive, Waterford, NY. Each is a dimethicone (polydimethylsiloxane) at 60% in water.

The polyorganosiloxane fluids are also commonly polydiorganosiloxanes and are referred to as “silicone oils” or “silicones” and are distinguished from silicone elastomers and resins, which are more thoroughly cross-linked than silicone oils. These example silicones can be linear or branched. Various naming conventions and nomenclature that are essentially equivalent to this exemplary class of silicones, include, but are not limited to: dialkylpolysiloxane hydrolyzate; alpha-alkyl-omega-methoxypolydialkylsiloxane; polydialkyl silicone oil; poly(dialkyl-siloxane); alkyl end-blocked polydialkylsiloxane; polyoxy(dialkylsilylene), alpha-(trialkylsilyl)-omega-hydroxy; poly[oxy(dialkylsilylene)], alpha-[trialkylsilyl]-omega-[(trialkylsilyl)oxy]; and alpha-(trialkylsilyl)poly[oxy(dialkylsilylene)]-omega-alky. Some additional suitable examples also include dimethicone copolyol, dimethyl-polysiloxane, diethylpolysiloxane, high molecular weight dimethicone, mixed C1-C30 alkyl polysiloxane, phenyl dimethicone, dimethiconol, and mixtures thereof. The silicone compounds useful herein also include polyalkyl or polyaryl siloxanes. The alkyl or aryl groups substituted on the siloxane chain (R) or at the ends of the siloxane chains can have any structure as long as the resulting silicone remains fluid at or around room temperature. Suitable R groups include hydroxy, methyl, methoxy, ethyl, ethoxy, propyl, propoxy, phenyl, methylphenyl, phenylphenyl, aryl and aryloxy. One or more R groups on the silicon atom may represent the same group or different groups, or any combination thereof.

Suitable silicone compounds are polydimethylsiloxane, polydiethylsiloxane, polymethylphenylsiloxane, polyalkylarylsiloxane, polyethyleneoxydialkylsiloxane, polypropyleneoxydialkylsiloxane, polydialkylcyclosiloxane, and mixtures thereof. Polydimethylsiloxane, which is also known as dimethicone, is suitable and readily available in many forms and grades, including for example, edible grades suitable for use in compositions for food contact usage. The polyalkylsiloxanes that can be used include, for example, polydimethylsiloxanes.

Other suitable polydiorganosiloxanes include polyalkylaryl siloxane fluids containing one or more alkyl or alkylaryl substituents can also be used, for example, and include, but are not limited to polymethylphenylsiloxanes, poly[(dimethylsiloxane)/methylvinyl-siloxane)], poly[(dimethylsiloxane)/(diphenylsiloxane)], poly[(dimethylsiloxane)/-(phenylmethylsiloxane)], poly[(dimethylsiloxane)/(diphenylsiloxane)/-(methyl-vinylsiloxane)], and mixtures thereof.

Higher molecular weight silicones, including silicone gums and resins, may be used in accordance with the present disclosure and include polydiorganosiloxanes with a molecular mass of between 200,000 and 5,000,000, used alone or as a mixture in a solvent chosen from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof. The silicones can be linear or branched, and can be modified by chemical groups to provide additional properties.

Organo-modified silicones potentially useful in the present disclosure are silicones as defined above, containing in their general structure one or more organofunctional groups directly attached to the siloxane chain or attached via a hydrocarbon-based radical. Examples include silicones containing: a) polyethyleneoxy and/or polypropyleneoxy groups; b) (per)fluoro groups, for instance trifluoroalkyl groups; c) hydroxyacylamino groups; d) thiol groups; e) carboxylate groups; f) hydroxylated groups; g) alkoxylated groups containing at least 12 carbon atoms; h) acyloxyalkyl groups containing at least 12 carbon atoms; i) quaternary ammonium groups; j) amphoteric or betaine groups; and k) bisulphite groups. If used, modified silicone could provide added protection and gloss to treated surfaces.

The polyorganosiloxanes in the composition are also believed to provide a soil, oil, dirt and grime resistant coating upon the surface of the treated materials to enhance their resistant to staining and soiling, water and microbial growth. In one embodiment of the present disclosure, the polyorganosiloxane includes one or more of a silicone selected from polydimethylsiloxane, polydiethylsiloxane, polymethylphenylsiloxane, polyalkylarylsiloxane, polyethyleneoxydialkylsiloxane, polypropyleneoxydialkylsiloxane and polydialkylcyclosiloxane.

In suitable embodiments, the total concentration of silicone fluids in the silicone package can range from about 0.1% by weight to about 45% by weight, or about 0.2% by weight to about 40% by weight, or alternatively about 1% by weight to about 30% by weight of the inventive composition.

The disinfectant package used in the solutions of the present disclosure typically include quaternary ammonium compounds. Quaternary ammonium compounds, also known as “quats”, typically comprise at least one quaternary ammonium cation with an appropriate anion. Quaternary ammonium compounds will generally have the formula (I):

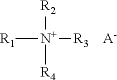

The groups R1, R2, R3 and R4 can vary within wide limits and examples of quaternary ammonium compounds that have anti-microbial properties will be known to the person of ordinary skill in the art. A- is a monovalent anion or one equivalent of a polyvalent anion of an inorganic or organic acid. Suitable anions A- are in principle all inorganic or organic anions, in particular halides, for example chloride or bromide, carbonates, bicarbonates, carboxylates, sulfonates, phosphates or a mixture thereof.

Each group R1, R2, R3 and R4 may, for example, independently be a substituted or unsubstituted and/or straight chain or branched and/or interrupted or uninterrupted alkyl, aryl, alkylaryl, arylalkyl, cycloalkyl, (aromatic or non-aromatic) heterocyclyl or alkenyl group. Alternatively, two or more of R1, R2, R3 and R4 may together with the nitrogen atom form a substituted or unsubstituted heterocyclic ring.

The total number of carbon atoms in the groups R1, R2, R3 and R4 must be at least 4. Typically the sum of the carbon atoms in the groups R1, R2, R3 and R4 is 10 or more. In a one aspect of the present invention, at least one of the groups R1, R2, R3 and R4 contains from 8 to 18 carbon atoms. For example, at least 1 and up to 4 of R1, R2, R3 and R4 can contain from 8 to 18 carbon atoms, typically 10 to 16 carbon atoms.

Suitable substituents for the groups R1, R2, R3 and R4 may be selected from the group consisting of alkyl, substituted alkyl, alkenyl, substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl. Each of the substituents on the substituted groups may be F, Br, I, —OR′, —NR′R″, —CF3, —CN, —NO2, —C2R′, —SR′, —N3, —C(═O)NR′R″, —NR′C(O)R″, —O(CR′R″)rC(═O)R′, O(CR′R″)rNR′C(O)R′, —(CR′R″)rNR″SO2R′, —OC(O)NR′R″, —NR′C(O)OR″, —SO2R′, —SO2NR′R″, and —NR′SO2R″, wherein R′ and R″ are individually hydrogen, C1-C8 alkyl, cycloalkyl, heterocyclyl, aryl, or arylalkyl, and r is an integer from 1 to 6, or R′ and R″ together form a cyclic functionality.

Exemplary quaternary ammonium compounds include dialkyldimethyl ammonium compounds, and alkyldimethylbenzyl ammonium compounds, where the alkyl groups 8 to 18 carbon atoms. Specific quaternary ammonium compounds include di C8-10 alkyldimethylammonium salt and a benzyl C12-16 alkyldimethyl ammonium salt. In one embodiment of the invention a mixture of include di C8-10 alkyldimethylammonium salt and a benzyl C12-16 alkyldimethyl ammonium salt. In a particular embodiment, the di C8-10 alkyldimethylammonium salt is a di C8-10 alkyldimethylammonium chloride and the benzyl C12-16 alkyldimethyl ammonium salt is a benzyl C12-16 alkyldimethyl ammonium chloride.

Quaternary ammonium compounds are commercially available, for example, from Lonza, Inc. (Allendale, N.J) and 3M Company (Saint Paul, MN). Particular suitable quaternary ammonium compounds include Concentrate 40A from 3M and Bardac 50 Concentrate from Lonza, each a blend of 4 quaternary components-Alkyl Dimethyl Benzyl Ammonium Chloride, Octyl Decyl Dimethyl Ammonium Chloride, Dioctyl Dimethyl Ammonium Chloride, Didecyl Dimethyl Ammonium Chloride. Other similar quat compounds are also suitable.

In suitable embodiments, the total concentration of quaternary ammonium compounds in the disinfectant package can range from about 0.3% by weight to about 1.2% by weight, or about 0.35% by weight to about % 1.1 by weight, or alternatively about 0.39% by weight to about 1% by weight of the disinfectant composition.

In lieu of or in addition to the quaternary ammonium compounds, the disinfectant package may include at least one of a hydrogen peroxide and thymol-based disinfectants. One suitable thymol-based disinfectant, thymol oil (also known as 2-isopropyl-5-methylphenol), is an essential oil extracted from various plants and has been used in the industry as an antibacterial product that has recently been approved to kill certain coronaviruses responsible for COVID-19 pandemic by the US Environmental Protection Agency.

In some embodiments, the disinfecting composition may include charge stabilizers to impart or improve the stability of the silicone package, particularly if the package includes a dimethicone silicone emulsion. Suitable charge stabilizers include NaCl, available from various sources. The charge stabilizer may be present, if used, at about 0.5% by weight to about 1.5% by weight of the disinfectant car care composition.

A solvent may be included in the disinfecting car care compositions of the present disclosure to assist in removing dirt, grease, and other unwanted impurities from the surface to be treated. The particular solvent employed in may be selected depending on the particular end use application, and particularly on the type of surface to be treated. In addition, the solvent may serve to help solubilize non-water soluble or poorly water soluble adjuvants, such as ultraviolet light (UV) absorbers, fragrances, perfumes and the like, for the purpose of preventing separation of these ingredients in the inventive compositions. Suitable solvents include both hydrophilic and hydrophobic compounds, generally comprising solvents that are water soluble, water-miscible as well as water insoluble and water-immiscible compounds. Mixtures of any solvent may optionally be employed. A suitable solvent for use in the compositions of this disclosure is an isopropyl alcohol. Solvents may be present at level between about 0-0.5%, based on total weight of the composition.

Water is typically a predominant ingredient in the disinfecting car care composition of the present disclosure. The water may be any suitable grade of water, though is preferably water purified through reverse osmosis or other purification technique. Water is typically present at a level of greater than 50% by weight, based on total weight of the composition. The level of water may include water added during the processing of disinfecting compositions as well as water added by incorporation of any silicone or other emulsion.

The disinfectants of the present disclosure may further include other common additives typically employed in car care formulations, including but not limited to preservatives, fragrances, UV blockers (including UV absorbers and UV scatters), stabilizers other than charge stabilizers, solubilizing materials other than solvents, lubricants, moisturizers, wetting agents, leveling agents, pH adjusters, buffers, thickeners, spreading agents, dyes, colorants, pigments, and odor absorbers.

Preservatives, when used, include, but are not limited to, mildewstat or bacteriostat, methyl, ethyl and propyl parabens, short chain organic acids (e.g., acetic, lactic and/or glycolic acids), bisguanidine compounds, and/or short chain alcohols (e.g., ethanol and/or isopropyl alcohol).

Suitable, exemplary commercially available preservatives include Acticide LA1209 from Thor Group, DANTOGUARD and DANTOGUARD PLUS from Lonza, Inc, KATHON GC, a 5-chloro-2-methyl-4-isothiazolin-3-one, KATHON ICP, a 2-methyl-4-isothiazolin-3-one, and blends thereof (available from DuPont, Wilmington DE), and a BPE-1 blend of phenoxyethanol and BIT from Thor Group.

Suitable, exemplary commercially available UV blockers include UNISORB 735 and UNISORB 720, (each from French Color & Fragrance, Englewood N.J.) and UVINUL N539 from BASF Corporation (Shakopee, MN).

Suitable, exemplary odor absorbents include TEGO Sorb A30, a zinc salt of ricinolic acid, available from Evonik/Goldschmidt (Essen, Germany).

Objects and advantages of this disclosure are further illustrated by the following non-limiting examples, but the particular materials and amounts thereof recited in these examples, as well as other conditions and details, should not be construed to unduly limit this disclosure. °=degree, in.=inch, lb.=pound, min.=minute, g=grams, ° C.=degrees Celsius, RH=relative humidity, mL=milliliters, %=percentage, L=liter, rpm=revolutions per minute.

### EXAMPLES

Unless otherwise noted, all parts, percentages, ratios, etc. in the Examples and the rest of the specification are by weight.

TABLE 1
Materials

| Abbreviation | Description and Source |
|---|---|
| Silicone Emulsion | A Silicone fluid emulsion consisting of 50-90% silicone fluid |
| | ranging in viscosity of 350 cps-1000 cps. |
| | XIAMETER 349 - Dow |
| | SM 2163 - Momentive |
| | Purpose-imparts gloss and barrier protection. Enhances leather |
| | surface |
| RO water | Water source for production utilizing Reverse Osmosis purification |
| | Purpose- diluent |
| Aloe 10X | Aloe Vera gel 10:1 |
| | From BioOrganic Concepts |
| | Purpose- Moisturizer |
| AMP 95 | 2-amino- 2methyl-1-propanol (95%) |
| | From Angus Chemical Co |
| | Purpose- Ph adjuster |
| NaCl | Sodium Chloride |
| | From Morton Co. |
| | Purpose- stabilize ionic charge for silicone emulsion stability |
| Kathon CG | Methylchloroisothiazolinone (CMIT) and Methylisothiazolinone |
| | (MIT) |
| | From DuPont |
| | Purpose- Preservative |
| Fragrance | Water soluble fragrance oil or blend |
| | From French Color and Fragrance Co. |
| | Purpose- fragrance |
| Unisorb 735 | UV absorbing/blocking polymer water soluble or blend |
| | From French Color and Fragrance Co. |
| | Purpose- UV blocker |
| Concentrate 40A | Blend of quaternary ammonium chlorides |
| | From 3M Company |
| | Purpose - disinfectant |
| Isopropyl Alcohol | Anhydrous IPA |
| | From Dow |
| | Purpose- mild cleaning action and solution solubilizer |
| Tego Sorb A-30 | Zinc Salt of Ricinolic Acid and solubilizer |
| | From Evonik/Goldschmidt |
| | Purpose- Odor absorbent & solubilizer |
| Acusol A820 | Acrylic Polymer |
| | From Dow |
| | Purpose: Thixotropic additive |
| LC250DC | Blend of quaternary ammonium chlorides From Lonza |
| Bardac BD28G | Blend of quaternary ammonium chlorides From Lonza |
| Thymol Oil | Amazon, Disinfectant, |
| Sodium Lauryl Sulphate | Surfactant, Sigma-Aldrich |
| Citric Acid | Sigma-Aldrich, adjust pH |
| Sodium Hydroxide | Sigma-Aldrich, Adjust pH |

### Vehicle Interior Surface Testing

Products were misted on all test surfaces with a spray bottle, allowed 3-5 minutes of dwell time and then wiped evenly with a microfiber towel. Also applied directly to towel and wiped on surface to test alternative application method. Vehicles tested include a 2014 Hyundai Azera, 2019 BMW X3, 2018 Toyota 4Runner, 2019 Mercedes C300, 2020 Ford Expedition. Surface tested, for each vehicle, include the dash, the seats, the console, the door panels, the interior panels, and a leather wrap. The composition of each surface is catalogued in Table 2 below.

TABLE 2
Interior surfaces composition

| | | | | Door | Interior | |
|---|---|---|---|---|---|---|
| | Dash | Seats | Console | panels | panels | misc |
| Hyundai | composite | leather | Plastic/ | plastic | Composite/ | Leather |
| | | | composite | | leather | wrap |
| BMW | composite | leather | Plastic/ | Composite/ | Plastic/ | Leather |
| | | | woodgrain | leather | leather | wrap |
| Toyota | composite | leather | Plastic/ | Plastic/ | Leather/ | Leather |
| | | | leather | composite | composite | wrap |
| Mercedes | Composite/ | leather | Plastic/ | Plastic/ | leather | Leather |
| | Leather | | leather | leather | | wrap |
| Ford | composite | leather | plastic | composite | Composite/ | Leather |
| | | | | | Leather | wrap |

### Antimicrobial Assay Protocol

An in vitro, time-kill assay was used to determine the effectiveness of the antimicrobial solutions. This test shows the ability of an antimicrobial agent to reduce number of viable cells of the chosen microorganisms during a chosen contact time.

Microorganisms tested in these experiments were Pseudomonas aeruginosa ATCC 9027 and Staphylococcus aureus (ATCC 6538). Microorganisms were grown on suitable agar and inoculums prepared in Butterfield's phosphate buffer with an optical cell density of approximately 3.0 McFarland. Antimicrobial solution (test sample, 2.7 ml) was combined with inoculums (300 μl) and vortex to achieve good mixing. After determined time point (3 min and 5 min), solution was neutralized with DE (Dey-Engley) broth to stop antimicrobial activity. Neutralized samples were further serial diluted and plated onto 3M Aerobic Petrifilm. After incubation, petrifilm plates were counted and number of surviving microorganisms expressed in colony forming units (CFU) estimated. Test control was prepared by combining Butterfield's phosphate buffer with inoculums and carried out in the same manner as a test sample.

### Preparation of Exemplary Compositions

Compositions for Examples 1-4 and 7 were prepared according to the following method.
1) Charge RO water to the mixing vessel and begin light agitation
2) Add Kathon CG biocide and mix until dispersed and clear
3) Add in order to the mixing vessel: Isopropyl Alcohol, Concentrate 40A. Mix until clear
4) Add in order to the mixing vessel: Silicone Package, Unisorb 730, Tego Sorb A30, fragrance. Mixed until uniform with light to moderate agitation.
5) Add AMP 95 to adjust PH. Final formulas should spray easily form trigger sprayer with Target Viscosity 10-100 cps (Brookfield DV1) and PH-8.0-9.5

For Examples 5 and 6, the silicone package includes both SM2163 emulsion and a 350 cSt silicone fluid from Dow.

Compositions for Examples 5, 6, 8, and 9 were prepared according to the following method:
1) Charge RO water to the mixing vessel and begin light agitation
2) Add Kathon CG, and NaCl-mix until dispersed and clear
3) Add Concentrate 40A. Mix until clear
4) Add in order to the mixing vessel: Silicone Package, Unisorb 735, Aloe 10X, fragrance. Components were mixed until uniform with light to moderate agitation.
5) Add AMP 95 if PH adjustments are needed, based on PH-8.0-9.5 target.
Target Viscosity 10-300 cps (Brookfield DV1) PH-8.0-9.5

Example 10 could be prepared according to the following method:
1) Charge RO water to the mixing vessel and begin light agitation
2) Add Kathon CG and NaCl-mix until dispersed and clear
3) Add Concentrate 40A. Mix until clear
4) Add in order to the mixing vessel: Silicone Package, Unisorb 735, Aloe 10X, fragrance. Mix until uniform with light to moderate agitation.
5) QC PH and visual inspection-add AMP 95 in formulas where PH adjustments are needed target (8.5-9.5)
Target viscosity 10-300 cps (Brookfield DVI) Target Ph 8.0-9.5
Comparative Example 1 is G109, a Gold Class Rich Leather Cleaner & Conditioner available from Meguiar's Inc., Irvine, CA. Comparative Example 2 is G136, Quick Interior Detailer Cleaner from Meguiar's Inc., Irvine, CA.

The formulation of the disinfecting compositions in Examples 1-21 are provided in Table 3. Unless otherwise noted, values in these Examples are reported in wt. %.

TABLE 3
Formulations for Examples 1-8

| | Example | Example | Example | Example | Example | Example | Example | Example |
|---|---|---|---|---|---|---|---|---|
| | 1 | 2 | 3 | 4 | 5 | 6 | 7 | 8 |
| Water (RO or DI) | 98.37% | 98.24% | 78.2% | 77.9% | 71.1% | 71.0% | 98.29% | 69.9% |
| AMP 95 | | | 0.20% | 0.20% | 0.10% | 0.10% | | |
| Sodium Chloride | | | | | | | | 1.00% |
| Isopropyl alcohol | 0.35% | 0.35% | | | | | 0.30% | |
| 40A Quat | 0.39% | 0.52% | 0.39% | 0.52% | 0.39% | 0.52% | 0.52% | 0.78% |
| Concentrate | | | | | | | | |
| 2163 silicone | 0.40% | 0.40% | 8.00% | 8.00% | | | 0.40% | |
| emulsion | | | | | | | | |
| DC349 silicone | | | | | 28.00% | 28.00% | | 28.00% |
| emulsion | | | | | | | | |
| 350 cst silicone | | | 13.0% | 13.0% | | | | |
| fluid | | | | | | | | |
| colorant | | | 0.002% | 0.002% | | | | |
| Unisorb 720 | 0.10% | 0.10% | 0.050% | 0.050% | | | | |
| Unisorb 735 | | | | | 0.10% | 0.10% | 0.10% | 0.10% |
| Tegosorb A30 | 0.15% | 0.15% | | | | | 0.15% | |
| Aloe vera 10x | | | 0.20% | 0.20% | 0.20% | 0.20% | | 0.20% |
| dispersion | | | | | | | | |
| Kathon CG | 0.09% | 0.09% | 0.09 | 0.09% | 0.09% | 0.09% | 0.09% | 0.07% |
| Fragrance #609 | 0.15% | 0.15% | | | | | 0.15% | |
| Fragrance #602 | | | | | | | | |
| Total | 100.0% | 100.0% | 100.0% | 100.0% | 100.0% | 100.0% | 100.0% | 100.0% |

TABLE 3
Formulations for Examples 9-16

| | Example | Example | Example | Example | Example | Example | Example | Example |
|---|---|---|---|---|---|---|---|---|
| | 9 | 10 | 11 | 12 | 13 | 14 | 15 | 16 |
| Water (RO or DI) | 59.23% | 98.57% | 98.57% | 98.18% | 98.18% | 97.87% | 97.87% | 69.96% |
| Sodium Chloride | 1.00% | | | | | | | |
| Isopropyl alcohol | | 0.30% | 0.30% | 0.30% | 0.30% | 0.30% | 0.30% | |
| 40A Quat | 0.78% | | | 0.78% | 0.78% | 0.78% | 0.78% | 1.57% |
| Concentrate | | | | | | | | |
| Lonza LC250DC | | 0.39% | 0.39% | | | | | |
| 2163 silicone | | 0.40% | | 0.40% | | | 0.40% | |
| emulsion | | | | | | | | |
| DC349 silicone | 38.50% | | 0.40% | | 0.40% | 0.40% | | 28.00% |
| emulsion | | | | | | | | |
| Unisorb 735 | 0.10% | 0.10% | 0.10% | 0.10% | 0.10% | 0.10% | 0.10% | 0.10% |
| Kathon CG | 0.09% | 0.09% | 0.09% | 0.09% | 0.09% | | | 0.07% |
| Proxel AQ | | | | | | 0.40% | 0.40% | |
| Fragrance #609 | | 0.15% | 0.15% | 0.15% | 0.15% | 0.15% | 0.15% | |
| Fragrance #627 | | | | | | | | 0.30% |
| Fragrance #602 | 0.30% | | | | | | | |
| Total | 100.0% | 100.0% | 100.0% | 100.0% | 100.0% | 100.0% | 100.0% | 100.0% |

TABLE 3
Cont.: Formulations for Examples 17-21

| | Example | Example | Example | Example | Example |
|---|---|---|---|---|---|
| | 17 | 18 | 19 | 20 | 21 |
| Water (RO or DI) | 71.09% | 59.24% | 59.07% | 59.44% | 60.57% |
| AMP 95 | | 0.0005 | 0.0005 | | |
| 40A Quat Concentrate | | 0.0052 | 0.0039 | 1.57% | |
| Lonza Bardac BD28G | 0.44% | | | | 0.44% |
| DC349 silicone | 28.00% | 0.4 | 40.00% | 0.385 | 38.50% |
| emulsion | | | | | |
| Unisorb 735 | 0.10% | 0.10% | 0.10% | 0.10% | 0.10% |
| Kathon CG | 0.07% | 0.09% | 0.09% | 0.09% | 0.09% |
| Fragrance #627 | 0.003 | | | | |
| Fragrance #602 | | | 0.003 | 0.003 | 0.30% |
| Total | 100.0% | 100.0% | 100.0% | 100.0% | 100.0% |

### Vehicle Interior Surface Testing Results

Compositions from Examples 3 and 4 were tested along with Comparative Example 1. Identical results were achieved through all compositions. The addition of the disinfectant package did not affect application, gloss, or cleaning any differently for Examples 3 and 4. Gloss levels were slightly higher for the Example 4, which was noticeable on several vehicles. Cleaning levels were slightly better for both Examples 3 and 4 (most noticeably on leather wrapped steering wheels).

### Antimicrobial Assay Results

Examples 1-21, and Comparative Example 1 and Comparative Example 2 were tested according to the Antimicrobial Assay Protocol. Staph aureus ATCC 6538 and Pseudomonas aeruginosa ATCC 9027 were chosen because they represent Gram positive and Gram-negative bacteria.

TABLE 4
Log Reduction of Staph Aureus ATCC 6538 and Pseudomonas aeruginosa

| | Log Reduction on | | | | Log reduction on | | | |
|---|---|---|---|---|---|---|---|---|
| | Staph Aureus ATCC 6538 | | | | Pseudomonas aeruginosa | | | |
| | 0.5 | 3 | 5 | 10 | 0.5 | 3 | 5 | 10 |
| Sample | minute | minutes | minutes | minutes | minute | minutes | minutes | minutes |
| Comparative Example 1 | n/a | n/a | n/a | 0 | n/a | n/a | n/a | 0 |
| Comparative Example 2 | | | | | | | | |
| Example 1 | n/a | 6 | n/a | n/a | n/a | 4.2 | n/a | n/a |
| Example 2 | n/a | 6 | n/a | n/a | n/a | 6 | n/a | n/a |
| Example 3 | n/a | 6 | n/a | n/a | n/a | 1.31 | n/a | n/a |
| Example 4 | n/a | 6 | n/a | n/a | n/a | 4.41 | 5.67 | n/a |
| Example 8 | n/a | 2.78 | 3.54 | n/a | n/a | 2.78 | 3.53 | n/a |
| Example 9 | n/a | n/a | 2.64 | n/a | n/a | n/a | 1.8 | n/a |
| Example 10 | n/a | 6.2 | 6.1 | n/a | n/a | 5.9 | 6 | n/a |
| Example 11 | n/a | 6.1 | 6.1 | n/a | n/a | 5.9 | 6 | n/a |
| Example 12 | 6.2 | 6.2 | 6.2 | n/a | 6.3 | 6.3 | 6.3 | n/a |
| Example 13 | 6.2 | 6.2 | 6.2 | n/a | 6.3 | 6.3 | 6.3 | n/a |
| Example 14 | 6.3 | n/a | n/a | n/a | 6.0 | n/a | n/a | n/a |
| Example 15 | 6.3 | n/a | n/a | n/a | 6.0 | n/a | n/a | n/a |
| Example 16 | n/a | 6.2 | 6.2 | n/a | n/a | 6 | 6 | n/a |
| Example 17 | n/a | 6.2 | 6.2 | n/a | n/a | 6 | 6 | n/a |
| Example 18 | n/a | n/a | n/a | n/a | n/a | 0 | n/a | n/a |
| Example 19 | n/a | 1.4 | n/a | n/a | n/a | 0 | 0.1 | n/a |
| Example 20 | n/a | 6.2 | 6.2 | n/a | n/a | 5.9 | 5.7 | n/a |
| Example 21 | n/a | 6.2 | 6.2 | n/a | n/a | 4 | 4.1 | n/a |

A stable formulation with Thymol oil is provided as Example 22 in Table 5

TABLE 5
Formulations for Example 22

| 2RM | Weight % |
|---|---|
| Water (DI) | 97.88 |
| Isopropyl alcohol | 0.40 |
| Thymol Oil | 0.25 |
| 2163 silicone emulsion | 0.49 |
| Sodium Lauryl Sulphate | 0.20 |
| Citric Acid/Sodium Hydroxide | adjust pH to 4.5 |

| | 3 min Log reduction on | 3 min log reduction on |
|---|---|---|
| Sample | Staph Aureus ATCC 6538 | Pseudomonas aeruginosa |
| Example 22 | 6.16 | 6.13 |

The disinfecting car care compositions of Examples 22 exhibited complete kill and antimicrobial properties.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. A disinfecting composition, the composition including:
a silicone package, water, and a disinfectant package.

2. A disinfecting composition of claim 1, wherein the silicone package includes one or more silicone fluid emulsions.

3. The disinfecting composition of claim 2, wherein the silicone fluid emulsion is non-ionic.

4. The disinfection composition of claim 2, wherein the silicone fluid emulsion includes one or more polyorganosiloxanes.

5. The disinfecting composition of claim 2, wherein the silicone fluid emulsion is made from a silicone fluid having a viscosity of between about 350 cSt and 1000 cSt.

6. The disinfecting composition of claim 1, wherein the silicone fluid emulsion includes a dimethicone at 60% by weight of the total emulsion.

7. The disinfecting composition of claim 1, wherein the disinfectant package includes one or more quats.

8. The disinfecting composition of claim 1, wherein the composition exhibits a 3 min Log reduction on Staph aureus ATCC 6538 of at least 6 according to the Antimicrobial Assay Protocol.

9. The disinfecting composition of claim 1, and further comprising one or more of preservatives, fragrances, UV blockers, stabilizers, solubilizing materials, lubricants, moisturizers, wetting agents, leveling agents, pH adjusters, buffers, spreading agents, dyes, colorants, pigments, and odor absorbers.

10. The disinfecting composition of claim 9, wherein the stabilizer includes a charge stabilizer to stabilize an ionic charge in the silicone package.

11. The disinfecting composition of claim 10, wherein the charge stabilizer is sodium chloride.

12. A method of disinfecting an interior automotive surface, the method comprising:
applying the composition of claim 1 to the surface,
allowing the composition to dwell on the surface for at least thirty seconds, and
wiping the surface.
